(19) United States
(12) Patent Application Publication
(10) Pub. No.: US 2026/0191475 A1
(43) Pub. Date: Jul. 9, 2026

(54) DEVICES FOR PROCESSING MEASUREMENTS TAKEN BY A SENSOR WORN BY A PERSON

(71) Applicant: Panoramic Digital Health, Saint-Pierre-de-Chartreuse (FR)

(72) Inventors: Derek Hill, Saint-Pierre-de-Chartreuse (FR); Luke Tonin, Oxford (GB)

(21) Appl. No.: 19/555,673

(22) Filed: Mar. 3, 2026

Related U.S. Application Data
(63) Continuation of application No. 18/001,127, filed on Jun. 7, 2021.

(30) Foreign Application Priority Data
Jun. 8, 2020 (FR) FR2005976

(51) Int. Cl. A61B 5/00; A61B 5/0205; A61B 5/024; A61B 5/11

(52) U.S. Cl. CPC A61B 5/7267; A61B 5/02055; A61B 5/02438; A61B 5/1118; A61B 2560/0223

(57) ABSTRACT

A wearable device includes a measurement sensor configured to acquire raw measurements representative of movement or a physiological characteristic of a user. An electronic acquisition circuit is connected to the sensor and is configured to control acquisition and/or pre-processing of the raw measurements so each raw measurement, or pre-processing thereof, is controlled by an acquisition parameter that is stored in one hardware control register physically implemented in the electronic acquisition circuit, wherein a specified value is assigned to the acquisition parameter. The device is connected to a central processing unit (CPU) configured to receive the raw or pre-processed measurements to estimate a state of the user. The central processing unit is configured to periodically read a stored value of the acquisition parameter, compare the stored value with the specified value, and determine conformity. When non-conformity is detected, the central processing unit replaces the stored value with the specified value.

### CROSS-REFERENCE TO RELATED APPLICATIONS

This application is a continuation of U.S. patent application Ser. No. 18/001,127, filed Dec. 8, 2022, which is a national phase entry under 35 U.S.C. § 371 of International Patent Application PCT/EP2021/065127, filed Jun. 7, 2021, designating the United States of America and published as International Patent Publication WO 2021/249916 A1 on Dec. 16, 2021, which claims the benefit under Article 8 of the Patent Cooperation Treaty to French Patent Application Serial No. FR2005976, filed Jun. 8, 2020, the disclosure of each of which is hereby incorporated herein in its entirety by this reference.

### TECHNICAL FIELD

The technical field of the disclosure is processing of measurements taken by a sensor, in particular a nomadic sensor, worn/borne by a user. The measurements may be measurements of physiological characteristics or measurements of the movement of the user.

### BACKGROUND

Connected devices, such as smartphones, connected watches, smart patches or smart bandages, include sensors allowing characteristics of their user to be measured.

Such devices, for example, comprise actimetry sensors allowing precise characteristics relative to the movement of the user to be obtained. These sensors may notably be accelerometers, gyrometers, magnetometers and/or pressure sensors. These sensors may be grouped together to form inertial measurement units. A relatively simple and widespread application is, for example, counting steps. Certain connected devices allow physiological characteristics to be measured, for example heart rate and body temperature. Sensors for estimating blood oxygenation levels or for carrying out analysis of bodily fluids, of perspiration for example, have also been developed. When integrated into smartphones, smart watches, or other smart devices, these sensors benefit from a powerful software environment, comprising powerful computing capabilities coupled to wireless communication means.

Documents EP3474287, US20180078219 and WO2016204905 describe sensors that are intended to be worn/borne by a user, and that measure physiological characteristics of the user. The measurements taken by the sensors are subject to processing, so as to make corrections, or to eliminate aberrant values, for example outliers or measurements influenced by a movement of the user.

The measured characteristics may be interpreted by interpreting applications that are either implemented by the device, or installed on remote servers. It may, for example, be a question of interpreting applications related to well-being, sports coaching, monitoring the state of health of the user, or detecting occurrence of a situation putting the latter at risk. Generally, this type of application makes it possible to classify a user state: this state relates to his physical activity, his state of health, or a state likely to entail a risk. Such applications are constantly being developed. Mention may, for example, be made of applications allowing the following to be detected: driver drowsiness; the occurrence of an epileptic fit; the occurrence of symptoms of pathologies, such as, for example, Parkinson's disease, or sleep apnea; or even a fall or a loss of consciousness. There are also countless applications dedicated to monitoring the physical activity of a user.

Each sensor is controlled by dedicated software, usually called “firmware” or “embedded software.” This type of software allows the sensor to be parametrized. Over time, the dedicated software may be subject to updates or changes. Consequently, for a given type of sensor, the signal generated may vary depending on the version of the software controlling the sensor. However, the interpreting application, for which the signals are intended, does not necessarily allow for changes made to the controlling software. This puts the reliability of the data generated by the application at risk. Specifically, a signal, corresponding to a measurement of a given physical quantity, may be modified following update of the dedicated software of the sensor.

A similar problem arises when two identical sensors are respectively powered by two different power supplies. A change in the power supply may lead to a difference between the signals generated by the sensors, even though they should be identical.

The same problem arises when a given application receives measurements taken by different sensors, two different accelerometers for example, measuring the same physical characteristic.

Thus, the measurements delivered to a given application are subject to variability depending on the sensor used, and on the environment of the sensor: power supply, version of the firmware. The input data transmitted to an interpreting application may, therefore, vary, even though they correspond to the same physical quantity. Variability in the input data can lead only to variability in the interpretation delivered by the interpreting application, with consequences on the reliability of the results delivered by the latter. It will be understood that this question is particularly important when the interpreting application concerns the state of health of a patient, or detection of the occurrence of a risky situation. In such applications, it is necessary to limit as much as possible the occurrence of false positives (a pathological or risky state is wrongly detected) or false negatives (a pathological or risky state is not detected when it should be).

A method is provided that allows this problem to be addressed. The objective is to achieve better control of the signals measured by sensors and transmitted to interpreting applications such as described above.

### BRIEF SUMMARY

A first embodiment of the disclosure is a method for processing measurements, acquired by a measurement sensor, at various measurement times, the measurement sensor being:
integrated into a connected device, worn/borne by a user, the connected device being configured to be connected to a wireless communication network;
configured to acquire, at each measurement time, a measurement representative of a movement of the user or of a physiological characteristic of the user;
connected to an electronic control circuit configured to command acquisition of measurements by the sensor, and/or pre-processing of measurements acquired by the sensor, the electronic control circuit comprising at least one control register;
parametrized by at least one sensor parameter, each sensor parameter being stored in one control register of the electronic control circuit;

the method comprising:
acquiring measurements by means of the measurement sensor at various measurement times, so as to form a sequence of measurements;
transmitting data, established on the basis of the sequence of measurements, to an interpreting application, the interpreting application being programmed to estimate, on the basis of the transmitted data, a user state, the user state being selected from a plurality of predetermined states.

According to one embodiment, the method comprises, before acquisition of the measurements and/or during acquisition of the measurements:
verification of the conformity of the values of each sensor parameter with respect to the specified values, for each sensor parameter respectively, verification being considered to be:
   negative when at least one value of a sensor parameter does not conform with the value specified for the sensor parameter;
   positive when the value of each sensor parameter does conform with the value specified for the sensor parameter;
following a negative verification, updating each non-conforming sensor-parameter value, by replacing each non-conforming parameter value with a value specified for the parameter.

The method may comprise, in the course of acquisition of the measurements, a plurality of successive verifications of the conformity of the values of each parameter, the method being such that, following a negative verification, measurements acquired since a preceding positive verification and/or until a following positive verification are considered doubtful or invalid.

The method may comprise:
considering a standard sequence of measurements by the connected device;
transmitting the standard sequence of measurements to the interpreting application, this being done by the connected device;
comparing the measurement sequence transmitted to the interpreting application with the standard measurement sequence.

According to one embodiment, the method may comprise, following acquisition of a sequence of measurements:
processing each measurement of the sequence of measurements by means of a calibration model, so as to estimate, on the basis of each measurement, a reference measurement;
transmitting each estimated reference measurement to the interpreting application, the reference measurements then forming the data transmitted to the interpreting application;
the method being such that the calibration model is established in the course of a calibrating phase, comprising various calibration times, the calibrating phase comprising:
   i) acquiring calibration measurements by means of the measurement sensor, at the various calibration times, and obtaining reference measurements, at each calibration time, such that a reference measurement corresponds to each calibration measurement, at least one reference measurement being representative of a user state among the predetermined states;
   ii) on the basis of the reference measurements and of the calibration measurements, defining a calibration model, the calibration model being configured to estimate reference measurements on the basis of measurements acquired by the measurement sensor.

Each sensor measures a physical or chemical quantity. The method ensures that two measurements, corresponding to the same quantity, and respectively acquired by two different measurement sensors, lead, following processing by the calibration model, to the same reference measurement. A reference measurement may correspond to the measurement that would be obtained by a reference sensor.

In step i), the measurement sensor may be placed on a phantom, representative of at least one user state, the reference data being obtained from the phantom. The phantom may comprise a reference sensor, the reference sensor delivering reference measurements at each calibration time.

In step i), the calibration sensor may be placed on at least one test individual, the test individual also wearing/bearing a reference sensor, the reference sensor delivering a reference measurement at each calibration time.

Preferably, the calibration model implements a supervised artificial-intelligence algorithm, the supervised artificial-intelligence algorithm being parametrized in the course of the calibrating phase. The supervised artificial-intelligence algorithm may comprise a neural network. The neural network may be a recurrent neural network, and preferably a bidirectional recurrent neural network, and comprise an input layer and an output layer, such that in the course of processing of each measurement by the calibration model:
each measurement acquired by the measurement sensor forms one input layer of the neural network;
the output layer corresponds to the estimate of at least one reference measurement.

The method may comprise, between steps i) and ii), time synchronization of the calibration measurements with respect to the reference measurements.

The sensor may comprise at least:
a motion sensor, of the accelerometer and/or gyrometer and/or magnetometer type;
and/or a pressure sensor;
and/or an optical sensor;
and/or a chemical sensor;
and/or an electrical sensor;
a temperature sensor;
and/or a physiological sensor, configured to measure a physiological characteristic of the user.

The physiological characteristic may be selected from:
body temperature;
blood pressure;
a level of oxygen or carbon dioxide in the blood;
a characteristic of respiratory activity, a respiratory rate for example;
a characteristic of cardiac activity, a heart rate for example;
a level of perspiration;
a characteristic of muscular activity;
a characteristic of neural activity.

The user state may be selected from:
a state describing a physical activity of the user;
a state of stress;
a state of sleep or drowsiness;
a pathological state;
a symptomatic state;

a state corresponding to an occurrence of a situation putting the user at risk.

The user may be a living human being or a living animal.

The interpreting application may be implemented by a microprocessor integrated into the connected device, or by a microprocessor remote from the connected device and connected to the latter by a wired or wireless link.

A second embodiment of the disclosure is a connected device, intended to be worn/borne by a user, comprising a measurement sensor, the measurement sensor being configured to acquire, at various measurement times, a measurement representative of a movement of the user or of a physiological characteristic of the user, the measurement sensor being parametrized by sensor parameters, each sensor parameter being stored in a control register of a control circuit of the sensor, the device being configured to activate an interpreting application, the interpreting application being programmed to estimate, on the basis of the measurements acquired by the measurement sensor, a user state, the user state being selected from a plurality of predetermined states. The device may comprise a central unit. The central unit may be programmed to implement the steps of verification of the conformity of the value of each parameter and the optional step of updating each non-conforming parameter value, according to the first embodiment of the disclosure.

The central unit may be programmed to:
process each measurement using a calibration model such as described with reference to the first embodiment of the disclosure, so as to obtain, from each measurement, a reference measurement;
transmit each reference measurement to the interpreting application.

The operations relative to the conformity of the parameters and/or to application of the calibration model carried out by the central unit may be controlled by an interfacing application. The interfacing application forms an interface between the sensor and the interpreting application.

The disclosure will be better understood on reading the text describing examples of the embodiments that are given, in the rest of the description, with reference to the figures listed below.

### BRIEF DESCRIPTION OF THE DRAWINGS

FIG. 1 shows measurement sequences acquired simultaneously by two different sensors;

FIG. 2 schematically shows one example of a device allowing embodiments of the disclosure to be implemented.

FIG. 3A shows the main steps of a first component of a method according to embodiments of the disclosure.

FIG. 3B shows a process of periodic verification of a register of a measurement sensor.

FIG. 4A shows the main steps of a second component of a method according to embodiments of the disclosure.

FIG. 4B illustrates determination of a synchronization function.

FIGS. 4C, 4D and 4E schematically show a first recurrent neural network, a second recurrent neural network and a third neural network, respectively.

FIG. 4F gives an overview of a structure of a preferred calibration model.

FIG. 5A shows two experimental measurement sequences acquired from two differently parametrized sensors of the same type.

FIG. 5B shows the measurement sequences shown in FIG. 5A, and a measurement sequence to which a calibration model has been applied, so as to obtain an estimate of reference measurements.

FIG. 5C is a detail of FIG. 5B, between times t=300 and t=400 of the latter.

FIG. 6A shows two experimental measurement sequences acquired from two different sensors measuring the same physical quantity, in the present case, an acceleration.

FIG. 6B shows the measurement sequences shown in FIG. 6A, and a measurement sequence to which a calibration model has been applied, so as to obtain an estimate of reference measurements.

FIG. 7A shows two experimental measurement sequences acquired from two different sensors measuring the same physical quantity, in the present case, an acceleration.

FIG. 7B shows the measurement sequences shown in FIG. 7A, and a measurement sequence to which a calibration model has been applied, so as to obtain an estimate of reference measurements.

In FIGS. 1, 3B, 5A to 5C, 6A, 6B, 7A and 7B, the y-axis corresponds to the measurements and the x-axis corresponds to time.

FIG. 8A shows measurements delivered by a reference sensor and test sensor parametrized according to the same reference configuration.

FIG. 8B shows measurements delivered by the reference sensor and test sensor described with reference to FIG. 8A.

FIG. 8C shows the effect of a poor temporal parametrization (curve b) with respect to a reference configuration (curve a). FIG. 8C also shows detection of incorrect adjustment, and the effect of the correction induced by the embodiments of the disclosure.

FIGS. 9A and 9B illustrate device variants allowing the embodiments of the disclosure to be implemented.

### DETAILED DESCRIPTION

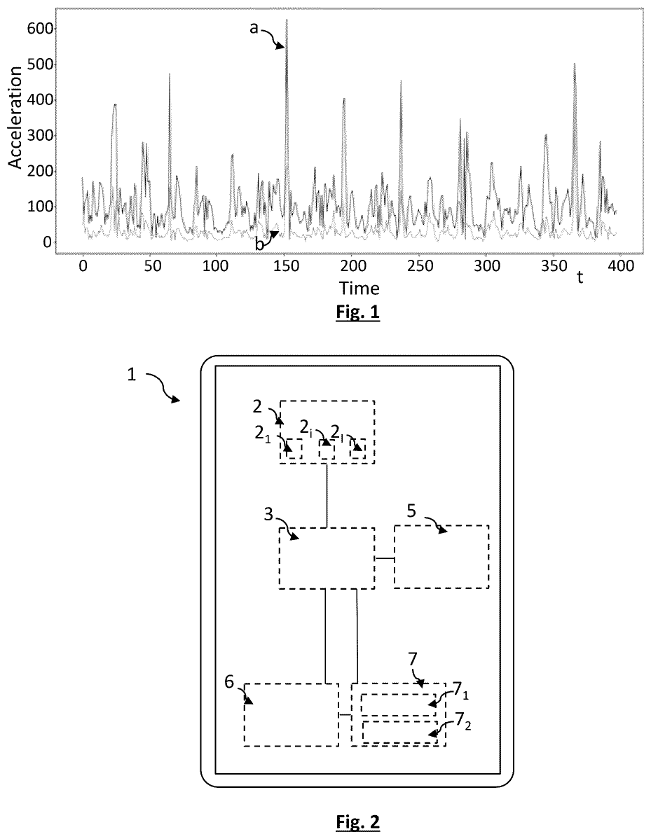

FIG. 1 shows acceleration measurements acquired, at various times, by two motion sensors placed on the same limb of a person. The measurements were obtained using two identical accelerometers, installed in two different inertial measurement units, the latter each being controlled by a pre-processing circuit, this pre-processing circuit comprising a low-pass filter.

More precisely, a different low-pass-filter cut-off frequency was applied to the measurements of each of the two accelerometers. This resulted in two curves (curve a and curve b). Each acceleration peak is correctly detected. However, there is a significant difference in the amplitude of the detected signals. In addition, it has been observed that the ratio between the amplitudes cannot be expressed by a simple multiplicative factor.

Embodiments of the disclosure described below allow this type of situation to be addressed. In the example described below, reference is made, non-limitingly, to a connected device 1 taking the form of a smartphone. The connected device 1 is intended to be borne by a user, the latter most often being a living human being or animal. The device 1 is notably intended to be placed against the body of the user, either through placement in the pocket of a garment or through application directly to the body of the user. The connected device 1 is configured to be connected to a wireless communication network, for example, and non-limitingly, a Wi-Fi or 4G or 5G or BLUETOOTH® network.

Embodiments of the disclosure are applicable to other types of connected devices, for example to a watch, or a tablet or a smart bandage or a patch. The device 1, which is shown in FIG. 2, comprises a measurement sensor 2, the latter being parametrized by various control registers 2₁, 2ᵢ . . . 2_{I}. The registers are integrated into an electronic control circuit forming part of the sensor. The electronic control circuit may comprise a control microprocessor, in which case the registers are integrated into a memory connected to the microprocessor. The function of the electronic control circuit is to control acquisition of measurements by the sensor and/or a pre-processing of measurements acquired by the sensor. By pre-processing, what is meant is measurement processing carried out in the sensor, by the electronic control circuit. Pre-processing is carried out prior to transmission of the measurements to an interfacing application described below. The measurement sensor 2, and the registers, are controlled by firmware 3. The device 1 comprises a central unit 5, for example a central processing unit (CPU), the latter forming an interface between the various components present in the device. The central unit 5 may notably control the measurement sensor 2 via the firmware 3. The central unit 5 may also be a microcontroller unit (MCU).

The measurement sensor 2 may be, or comprise, a motion sensor, for example an accelerometer, a magnetometer, or a gyrometer. It may, for example, be an inertial measurement unit. The measurement sensor 2 may be or comprise a pressure sensor, an optical sensor, a temperature sensor, a chemical sensor, a gas sensor, or an electrical sensor, an electrode for example. It may also be a physiological sensor, configured to determine a physiological characteristic of the user. By physiological characteristic, what is meant is, for example, and non-limitingly:
body temperature;
blood pressure, arterial pressure for example;
a characteristic of cardiac activity, for example heart rate or an inter-beat interval;
a respiratory characteristic, a respiratory rate for example;
a characteristic of muscular activity;
a characteristic of neural activity, for example a neural activity measured by an electrode;
a characteristic of perspiration, a level of perspiration for example;
a concentration of oxygen or carbon dioxide in the blood.

Generally, the measurement sensor 2 is configured to measure a physical or chemical quantity that depends on a state of the user.

The device 1 implements an interpreting application 6, which is stored in a memory. The interpreting application may be implemented by the central unit 5, and call upon measurements taken by the measurement sensor 2. According to one variant, the interpreting application is stored on a remote server that is linked to the device 1 by a link, preferably a wireless link.

Generally, the interpreting application 6 is supplied with at least one sequence of measurements, originating from at least one measurement sensor. By sequence of measurements, what is meant is a set of measurements respectively acquired at various measurement times, and in general successive measurement times. The term “interpreting” in “interpreting application” is understood to mean that the application takes into account the measurements taken by the measurement sensor to establish an interpretation thereof. As mentioned with reference to the prior art, it may be a question of an application aiming to determine a state of the user, among predetermined states. By way of predetermined states, mention may be made of a state of stress, of fatigue, of drowsiness, of physical activity, of alertness, of rest, of sleep, of a state considered to be pathological or potentially pathological, and of a symptomatic state. The interpreting application may allow an assessment of the physical activity of the user during a determined period to be established. It may also allow a potential risk to the user to be detected.

It is particularly envisaged to obtain characteristics of the user with a view to assisting with clinical examination or detection of the onset of a pathological state. Thus, it is important for the reliability of the interpreting application 6 to be maximized, by limiting occurrences of false positives or false negatives, as indicated with respect to the prior art.

The device 1 comprises an interfacing application 7 that is stored in a memory, and that is intended to process the measurements taken by the measurement sensor, before the measurements are sent to the interpreting application. The objective of the interfacing application is to remedy the faults described with reference to FIG. 1 and with respect to the prior art. It is a question of making it so that different sensors measuring, at the same moment, the same physical or chemical quantity, transmit comparable measurements to the interpreting application 6. In other words, it is a question of decreasing the variability in the data transmitted to the interpreting application 6.

The interfacing application comprises a first component 71, which is intended to interact with the measurement sensor 2, by way of the firmware 3 associated with the sensor. It further comprises a second component 72, which is intended to process the measurements taken by the sensor, with a view to transmitting processed measurements to the interpreting application. Thus, the interfacing application 7 forms an interface between the measurement sensor 2 and the interpreting application 6. It acts on the measurement sensor, so as to ensure that the parametrization of the latter corresponds to predefined specifications. It also acts on the data measured by the measurement sensor, so as to ensure standardization of the latter with respect to reference measurements.

The objective is for the measurements transmitted to the interpreting application 6 after processing by the interfacing application 7 to be able to be considered independent of the connected device 1 worn/borne by the user, and more precisely of the measurement sensor 2. In other words, the interfacing application is configured in such a way that two different measurement sensors 2 (for example, two accelerometers), belonging to two different devices 1 (for example, two mobile telephones), and subjected to identical conditions, transmit, after processing by the interfacing application, sequences of data that are identical, or that may be considered as such, to the interpreting application.

The interfacing application 7 also makes it possible to guard against variations affecting the measurements acquired by the sensor as a result of:
modification of the communication protocol between the measurement sensor 2 and the interpreting application 6;
change of the power supply used to power the device 1 comprising the measurement sensor 2;
update of the firmware of the sensor, or of any other application downloaded to the device 1 and liable to have an effect on the measurements taken by the measurement sensor 2.

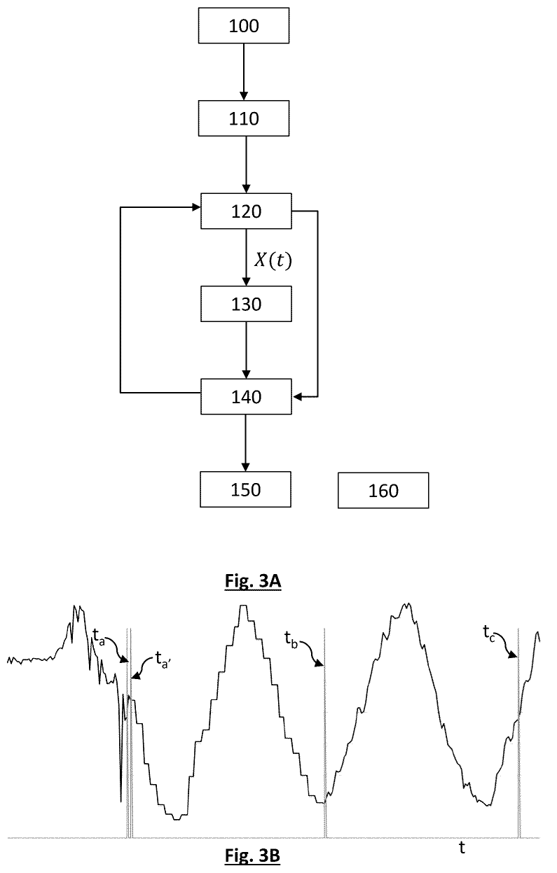

FIG. 3A schematically shows the main steps of a method implemented by the first component 71.
Step 100: Taking sensor parameters into account

In this step, the sensor parameters are taken into account. The sensor parameters are stored in one or more control registers 2; of the measurement sensor 2. As described above, the registers 2; are located in an electronic control circuit forming part of the measurement sensor 2. The sensor parameters may comprise acquisition parameters on which the way in which the measurement sensor 2 takes each measurement depends. It is a question of acquisition parameters governing the operation of the measurement sensor 2, and which are available in a specification. The acquisition parameters may notably depend on the end to which the measurements are taken. It is assumed that a specification containing the acquisition parameters of a sensor is available. An acquisition parameter may be selected from:
measurement frequency (sampling frequency);
an adjustment parameter of a frequency-domain filter applied to each measurement, for example a cut-off frequency of a low-pass filter or of a high-pass filter;
a duration of each sequence of measurements;
a time interval between two sequences of successive measurements.

The measurement sensor 2 may carry out a step of pre-processing the measurements, in which case a sensor parameter may be a pre-processing parameter governing pre-processing. Such pre-processing may be carried out in the measurement sensor 2 (for example, by the electronic control circuit) or by the firmware 3 associated with the sensor. A pre-processing parameter may comprise:
a parameter representing a threshold of detection of a movement;
a weighting factor assigned to a measurement, especially when a measurement is combined with other measurements. This is, for example, the case when a measurement is a component of a measured quantity, in which case the measurement may be combined with other measurements respectively representing other components.

Generally, the pre-processing is carried out in the measurement sensor 2, prior to transmission of the measurements to the interfacing application 7.

The sensor parameters may have been specified beforehand by the interpreting application. According to one embodiment, the specification, comprising the acquisition parameters, is transmitted by the interpreting application 6 to the interfacing application 7.
Step 110: Initializing the registers.

In this step, using the sensor parameters specified in step 100, the interfacing application 7 verifies and/or initializes the registers 2; of each measurement sensor 2, so that these registers conform with the specified sensor parameters.

Steps 120 to 150 are implemented at each measurement time of a sequence of measurements.
Step 120: Taking a measurement with the measurement sensor

In this step, the measurement sensor takes a measurement X(t) at a measurement time t, depending on the sensor parameters specified in the registers associated therewith. By measurement, what is meant is:
either raw measurements, acquired by the sensor;
or raw measurements having undergone pre-processing, for example calculation of an average, a moving average for example, or calculation of a median, or detection of an occurrence of particular measurements. A particular measurement may correspond to successive raw measurements the occurrence of which corresponds to a particular situation. For example, when the sensor is an accelerometer, a particular measurement may be a double tap exerted by the user on the device. The pre-processing carried out in the sensor may detect the occurrence of a double tap and convey the fact that this event occurred. This may correspond to a situation in which the user wishes to start a new sequence of measurements. The pre-processing may also make it possible to provide a measurement established on the basis of the raw measurements. It may, for example, be a question of a step count when the sensor is an accelerometer.
Step 130: Verifying the registers

In this step, the interfacing application 7 verifies that the content of each register 2; has not been modified. It is a question of ensuring that the registers 2; associated with the measurement sensor 2 have not been modified. It is not necessary for step 130 to be carried out at each measurement time. It may be implemented periodically, between a predetermined number of measurement times, for example every 5 minutes. When the registers have not been modified, and conform with the parametrization defined in step 110, measurements taken since the preceding verification are considered valid. When step 130 detects a modification of a register:
measurements taken since the preceding conforming verification and/or up to the following conforming verification are considered to be invalid, or doubtful (cf. step 150).
non-conforming registers are updated to conform with the specified parameters.
Step 140: Incrementing the measurement time and returning to step 120.
Step 150: Validating or invalidating measurements

Measurements X(t) carried out between two consecutive verifications, and considered to conform, are associated with a validity indicator (or label).

When a verification is non-conforming, measurements taken:
since the preceding conforming verification,
and/or until the next conforming verification,
   are considered invalid or doubtful, in which case they may be associated with an invalidity indicator. A measurement considered doubtful may be subject to a correction post-acquisition.

This prevents measurements considered invalid or doubtful from being subsequently taken into account by the interpreting application 6.

FIG. 3B illustrates the process of verification of the measurements that was described with reference to step 130. This figure shows measurements X(t) taken over time, describing a pseudo-sinusoid. The x-axis corresponds to time t and the y-axis corresponds to the measurements X(t) taken by the measurement sensor 2. Register verification such as described in step 130 is carried out periodically. In FIG. 3B, each verification has been represented by a vertical line. Three successive verifications, carried out at times tₐ, t_{b} and t_{c}, have been shown. The verification carried out at time tₐ detects a modification of one of the registers. A correction of each register is carried out at a time tₐ′, which is after the time tₐ. The verifications of register conformity carried out at times t_{b} and t_{c} do not detect an anomaly. The measurements taken between times tₐ and to are considered invalid, because the verification carried out at time tₐ detected an anomaly. As a result, measurements taken between times tₐ and t_{b} are assigned an invalidity indicator. The measurements taken between times to and the are valid.

According to one optional embodiment, the method comprises a step 160 of consideration of a standard sequence of measurements. Such a sequence may be downloaded from a public database or from a database linked to the interpreting application 6. It then forms an original standard sequence. In step 160, the interfacing application 7 transmits the standard sequence of measurements to the interpreting application 6. The original standard sequence is then compared with the sequence received by the interpreting application 6. The comparison may comprise determining a quality indicator indicative of the quality of the transmitted standard sequence. The quality indicator may be a quadratic error between the original standard sequence and the transmitted standard sequence. When the quality indicator crosses a predetermined threshold, the transmission is considered corrupt. A warning is then transmitted to the interpreting application 6, mentioning the fact that the data being transmitted to it are potentially corrupt. Otherwise, the transmission is considered correct. This step makes it possible to verify the quality of the protocol of transmission between the data processed by the interpreting application 6 and the interfacing application 7.

The main steps of the second component 72 of the interfacing application will now be described. In this step, the measurements delivered by the measurement sensor 2 are converted in such a way that, after conversion, the measurements correspond to the measurements that would have been acquired using a reference sensor. This step requires a calibration model to be established. The calibration model is parametrized in the course of a calibrating phase, carried out beforehand.

The objective of the calibrating phase is to establish the parameters of the calibration model, such that the calibration model may then be applied to each sequence of measurements delivered by the measurement sensor 2. The calibrating phase comprises a plurality of calibration times t′, forming one or more calibration sequences. The establishment of the calibration model requires reference measurements to be used. Three possibilities may be envisioned:
The reference measurements are taken by placing a sensor, of the same type as the measurement sensor with which the device 1 is equipped, on a phantom configured to vary the physical quantity. During the calibration sequence, the physical quantity varies within a coherent range of measurement values likely to be measured under real conditions. This possibility is particularly appropriate when the physical quantity measured is a movement. In this case, the reference values may be obtained by placing the measurement sensor on a robotic arm, playing the role of phantom, and the movement of which is controlled. The robotic arm may, for example, mimic the movement of a forearm, or of a leg, or of a thigh. The reference values are established depending on the programmed movements of the phantom. They may result from programming the movements of the phantom. It is also possible to use a phantom, the temperature of which, or the optical or chemical properties of which, are controlled, and variation as a function of time in the temperature of which or in the optical or chemical properties of which may be programmed.
The reference measurements are taken by placing a reference sensor, of the same type as the measurement sensor 2 with which the device 1 is equipped, on a phantom such as described in the preceding paragraph. A reference sensor, considered to provide accurate measurements, is also placed on the phantom.
Reference measurements are taken by equipping a population of test individuals with a reference sensor, considered to deliver accurate measurements. Each test individual in the population is also equipped with a measurement sensor 2. The population contains at least one test individual, and preferably a plurality of different test individuals. It is preferable for the population to comprise test individuals considered to be representative of the interpreting application 6 used. For example, when the interpreting application aims to determine a state of a user, it is preferable, in the course of the calibration, for at least one test individual of the population to be in a state targeted by the application (this corresponding to a true positive) and for at least one test individual to not be in a state targeted by the application (this corresponding to a true negative). A plurality of calibration sequences may be carried out for each test individual.

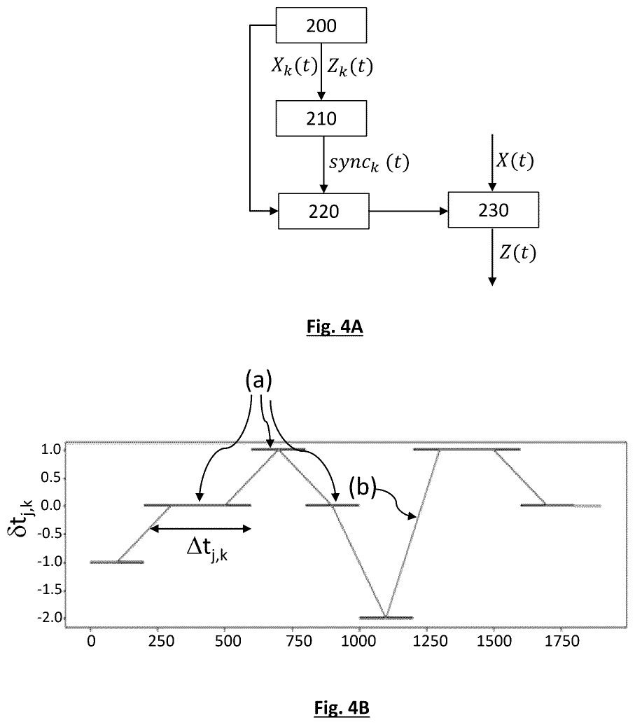

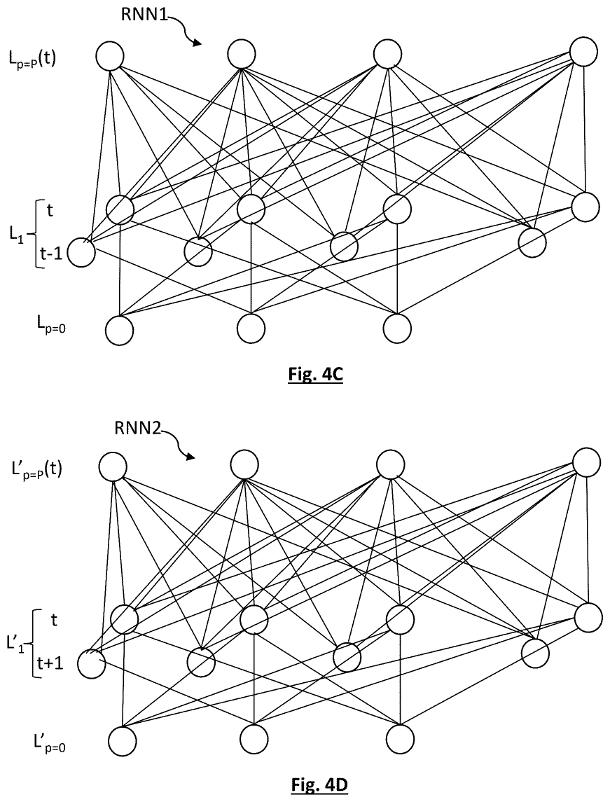

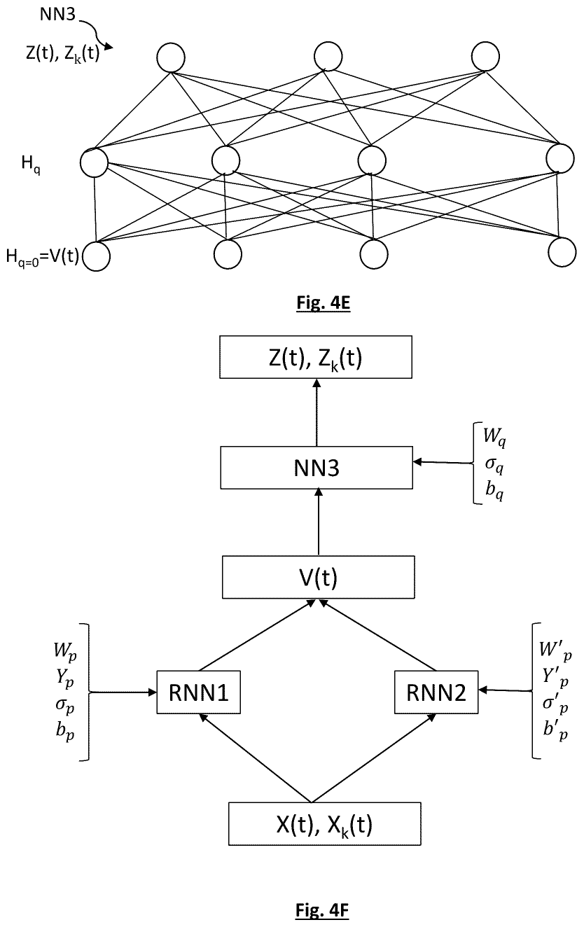

The calibrating phase comprises steps 200 to 230, which are illustrated in FIG. 4A.
Step 200: Acquiring the calibration measurements Xₖ(t) and the reference measurements Zₖ(t)

Over the course of the various calibration times t, the following are acquired:
calibration measurements Xₖ(t), taken by the measurement sensor 2, or by each measurement sensor 2, used in the calibration;
reference measurements Zₖ(t), taken, according to the circumstances, by the reference sensor if one is employed or by the operating parameters of the phantom.

The calibration measurements Xₖ(t) correspond to the measurements acquired with the measurement sensor 2 during the calibrating phase. The calibration measurements Xₖ(t) form different calibration sequences, the index k indexing each calibration sequence.
Step 210: Temporal synchronization of the calibration measurements and of the reference measurements.

In this optional step, the measurements acquired by the measurement sensor and the reference measurements are synchronized in time. It is a question of taking into account any temporal drift affecting the clocks respectively controlling the measurement sensor and the reference sensor if one is employed. The objective of this phase is to obtain the most precise synchronization possible between the reference measurements and the calibration measurements.

Step 210 may comprise establishing a synchronization function. The synchronization function may be obtained by dividing each calibration sequence k into/time segments Δt_{j,k} of short duration, for example of a duration comprised between 1 s and 5 s, and for example of a duration equal to 2 s. J is an integer designating the number of time segments into which a given calibration sequence is divided.

In each time segment Δt_{j,k}, a correlation coefficient between the reference measurements and the calibration measurements is determined taking into account various time shifts St between the reference measurements and the calibration measurements, including time shifts of zero. The time shifts in question are preferably small. It may be a question of time shifts δt lying on either side of 0. For example, δt=0; δt=+10 ms; δt=+20 ms; δt=+30 ms, etc. The time shift δt_{j,k} for which the correlation is maximum is determined. To each time segment Δt_{j,k} of a calibration sequence k then corresponds the time shift δt_{j,k} thus identified. A synchronization function sync is established for each calibration sequence k, on the basis of time segments Δt_{j,k} established, such that:

$${sync}_{k} ( t ∈ Δ {t}_{j , k} ) = t + δ {t}_{j , k} \qquad ( 1 )$$

The synchronization function syncₖ is then applied either to the reference measurements or to the calibration measurements, so as to obtain precise time synchronization. If Zₖ(t) and Xₖ(t) are the reference and calibration measurements acquired in the course of the same calibration sequence k, respectively, taking account of the synchronization function syncₖ then takes the form of a composition, such that:

$${Z}_{k} ( t ) \to {Z}_{k} ( t ) ∘ {sync}_{k} ( t ) \qquad ( 2 )$$

$$or$$

$${X}_{k} ( t ) \to {X}_{k} ( t ) ∘ {sync}_{k} ( t ) \qquad ( {2}^{′} )$$

FIG. 4B shows one example of variation as a function of time in the synchronization function (curve (a)). It may be seen that, according to expression (1), the synchronization function sync is discontinuous. Its value changes abruptly between each time segment δt_{j,k}. In order to avoid such discontinuities, step 210 may comprise an interpolation of the synchronization function such as expressed by (1), so as to obtain a continuous synchronization function. The interpolation is, for example, linear or polynomial. FIG. 4B shows an interpolated synchronization function. It corresponds to curve (b).
Step 220: Determining the calibration model.

Following step 210, or following step 200, if potential temporal drifts are neglected, the method comprises parametrization of a calibration model. The calibration model is configured to receive input data, corresponding to measurements X(t) acquired by the measurement sensor at a measurement time t. It is also configured to estimate reference measurements Z(t) on the basis of the input data. By reference measurements, what is meant are calibrated measurements, such that each calibrated measurement corresponds to one measured physical quantity, independently of the type or of the manufacturer of the sensor that recorded it, in a reference space. Thus, application of the calibration model may be considered to correspond to a change of frame of reference, between an initial frame of reference, corresponding to the measurement sensor, and a final frame of reference. In the final frame of reference, two measurements, corresponding to the same physical or chemical quantity, and respectively measured by two different measurement sensors, have the same value, or a value that may be considered to be identical.

When the calibration is carried out taking into account a reference sensor, the calibration model makes it possible to estimate, on the basis of a measurement taken by a measurement sensor 2, worn/borne by a user, a measurement that would have been delivered by the reference sensor, measuring the same physical or chemical quantity.

When the calibration is carried out without a reference sensor, but rather taking into account a phantom that controls the variation in a physical quantity, a reference value is considered to correspond to the physical quantity.

A measurement X(t) delivered by a measurement sensor 2 takes the form of a scalar or of a vector, the vector having various coordinates. A reference measurement Z(t), expressed in the final frame of reference, also takes a scalar form or the form of a vector. For a given physical quantity measured by various measurement sensors that are different from one another, the calibration model makes it possible to obtain reference values that are identical or that may be considered such.

The calibration model is established in the course of the calibrating phase, or training phase, using the calibration sequences and the reference sequences that are respectively associated with them. There are a number of ways of determining the calibration model. It is believed to be preferable for the calibration model to be established using a supervised artificial-intelligence algorithm, and, for example, using a neural network.

By supervised artificial-intelligence algorithm, what is meant is an algorithm parametrized during a training phase, in the course of which controlled input data and controlled output data are made available. In the present case, training is carried out using the calibration sequences as algorithm input data and the reference sequences as algorithm output data.

In the targeted application, the input and output data take the form of sequences of time-domain data. It is, therefore, particularly appropriate to use recurrent neural networks, and preferably bidirectional recurrent neural networks.

A first recurrent neural network RNN1, known to those skilled in the art, has been schematically shown in FIG. 4C. Recurrent neural networks are known to those skilled in the art, and are, in particular, used for applications related to voice recognition or machine translation. One example of application is given in the publication Cho K. “Learning Phase Representations using RNN Encoder-Decoder for Statistical Machine Translation,” ArXiv abs/1406.1078 (2014). In the example shown in FIG. 4C, the recurrent neural network comprises an input layer comprising the measurements Xₖ(t) taken by the measurement sensor at a calibration time t. The network also comprises a hidden layer Li and an output layer L_{P}. P is an integer strictly higher than 1 designating the number of layers in addition to the input layer. The input layer Xₖ(t) corresponds to each measurement acquired at a measurement time t. The dimension of Xₖ(t) corresponds to the dimension of each measurement. For example, when each measurement is a three-axis acceleration, the dimension of Xₖ(t) is equal to 3. When each measurement is a scalar, the dimension of Xₖ(t) is equal to 1.

For a sequence of input vectors Xₖ(t), with t∈[t₁ . . . t_{N}], a sequence of output vectors Xₖ(t), with t∈[t₁ . . . t_{N}], is obtained. Between the input layer and the output layer lies at least one hidden layer Lₚ, with 0<p<P. The layer L_{p=P} corresponds to the output layer.

In the case of recurrent neural networks, the value of the neurons of each hidden layer Lₚ(t), at each calibration time t, may be obtained:
either by considering an increasing chronological order, the layer corresponding to a time t−1 prior to the time t, this corresponding to the neural network RNN1 shown in FIG. 4C;
or by considering a decreasing chronological order, the layer corresponding to a time t+1 subsequent to the time t, this corresponding to the neural network RNN2 shown in FIG. 4D.

When an increasing chronological order is considered, this corresponding to the first neural network RNN1 schematically shown in FIG. 4C, the content Lₚ(t) of each layer of rank p, with 1≤p<P, is determined taking into account:
a transfer function σₚ: [symbol] , nₚ being the number of neurons in the layer Lₚ(t). The transfer function σₚ comprises nₚ elementary activation functions [symbol] . Each elementary function is generally non-linear and bounded. It is usually a question of a hyperbolic-tangent function or of a sigmoid function.
Wₚ is a first connectivity matrix defined for each layer Lₚ(t), allowing passage from the layer Lₚ₋₁(t) to the layer Lₚ(t), of dimension [nₚ₋₁, nₚ], and each term of which corresponds to one weight.
Yₚ is a second connectivity matrix defined for each layer Lₚ, allowing passage from the layer Lₚ(t−1) to the layer Lₚ(t), of dimension [nₚ, nₚ], and each term of which corresponds to one weight.
bₚ is a bias vector of the layer Lₚ, of dimension [1, nₚ].

The value of each layer Lₚ(t) is updated at each measurement time, and may be represented by a vector of dimension [1, nₚ]. It is obtained from a preceding layer Lₚ₋₁(t) at the measurement time, and from the layer Lₚ(t−1) of same rank p, at a time t−1 preceding the measurement time, using the expression:

$${L}_{p} ( t ) = {σ}_{p} [ {W}_{p} \times {L}_{p - 1} ( t ) + {b}_{p} + {Y}_{p} \times {L}_{p} ( t - 1 ) ] \qquad ( 3 )$$

During calibration, the input layer Lₚ₌₀ is Xₖ(t).

When the decreasing chronological order is taken into account, a second recurrent neural network RNN2, having a structure such as that shown in FIG. 4D, is used. The content of each layer L′ₚ(t) is calculated, at a time t, using the following expression, which is analogous to expression (3):

$${L}_{p}^{′} ( t ) = {σ}_{p}^{′} [ {W}_{p}^{′} \times {L}_{p - 1}^{′} ( t ) + {b}_{p}^{′} + {Y}_{p}^{′} \times {L}_{p}^{′} ( t - 1 ) ] \qquad ( 4 )$$

where W′ₚ, Y′ₚ, b′ₚ and σ′ₚ are a first connectivity matrix, a second connectivity matrix, a bias vector and an activation function analogous to the first connectivity matrix, the second connectivity matrix, the bias vector and the activation functions described with reference to expression (3), respectively. During calibration, the input layer L′ₚ₌₀ is Xₖ(t).

Expressions (3) and (4) assume an initialization, the value of each layer being considered to be equal to a predetermined value, or to a random value, at t=0.

The first neural network RNN1 and the second neural network RNN2 may comprise between 1 and 3 hidden layers, between the input and output layer. The number of neurons in each hidden layer may be comprised between 10 and 100 or more.

A vector V(t) is then formed by concatenating the output layers L_{p=P}(t) and L′_{p=P}(t). The vector V(t) is used as input datum H_{q=0} of a third neural network NN3, schematically shown in FIG. 4E. The third neural network NN3 comprises at least one hidden layer H_{q} and one output layer H_{q=Q}. The index q is an integer designating the rank of each layer. Q is an integer strictly higher than 1 designating the number of layers over and above the input layer. The third neural network NN3 may comprise between 1 and 3 hidden layers, between the input and output layer. The number of neurons in each hidden layer may be comprised between 10 and 100 or more.

The third neural network is a standard, i.e., non-recurrent, neural network, known to those skilled in the art. The content of each layer of rank q, with 1<q≤Q, is determined taking into account:
a transfer function σ_{q}: [symbol] , n_{q} being the number of neurons in the layer H_{q}. The transfer function σ_{q} comprises n_{q} elementary transfer functions [symbol] . Each elementary function is generally non-linear, bounded and differentiable. It is usually a question of a hyperbolic-tangent function or of a sigmoid function;
a connectivity matrix W_{q} defined for each layer H_{q}, allowing passage from the layer H_{q-1} to the layer H_{q}, of dimension [n_{q-1}, n_{q}], and each term of which corresponds to a weight.
a bias vector b_{q} of the layer H_{q}, of dimension [1, n_{q}].

The content of each layer H_{q} is calculated using the following expression:

$${H}_{q} = {σ}_{q} [ {W}_{q} \times {H}_{q - 1} + {b}_{q} ] \qquad ( 5 )$$

During calibration, the output layer H_{q=Q} of the third neural network NN3 is a reference measurement, also denoted Zₖ(t), corresponding to the calibration measurement Xₖ(t) forming the input layer of the first neural network RNN1 and of the second neural network RNN2.

FIG. 4F shows the relationship between the various neural networks, forming the calibration model.

The training phase allows the parameters of the activation model to be defined. It is a question, for each layer of order p≥1, of the parameters governing each neural network, i.e., of:
the matrices Wₚ, Yₚ, W′ₚ, Y′ₚ, W_{q};
the transfer functions σₚ, σ′ₚ, σ_{q},
the vectors bₚ, b′ₚ, b_{q}.

During learning, the input layer is formed from calibration measurements Xₖ(t) forming each calibration sequence, and the output layer is formed from reference measurements Zₖ(t) forming each reference sequence respectively corresponding to the calibration sequence used to define the input layer. Preferably, learning requires a high number of calibration sequences.

Learning consists in determining the parameters of the calibration model allowing the best possible estimate of each reference measurement to be obtained, from the calibration measurements. To do this, a cost function Cost may be used, the latter corresponding to a root-mean-square error (RMSE) or a mean-absolute error (MAE) between the reference measurements respectively measured and estimated by the calibration model. When based on the mean-absolute error, the parameters of the model are those minimizing a cost function Cost such as:

$$Cost = {\sum }_{t , k} \vert {Z}_{k} ( t ) - {\hat{Z}}_{k} ( t ) \vert \qquad ( 6 )$$

where Zₖ(t) and \hat{Z}ₖ(t) are a reference measurement obtained and estimated at a calibration time t of a calibration sequence k, respectively.

According to another embodiment, the calibration model is a linear model. It may be a question of a transfer matrix A, of dimension (nₓ, nₓ), where nₓ corresponds to the dimension of each measurement. The transfer matrix is learned, for example via minimization of a cost function such as described with reference to expression (6). The reference measurements may be such that \hat{Z}(t)=AX(t). The matrix A may be defined such that:

$$A = arg {min}_{A} {\sum }_{t , k} \vert {Z}_{k} ( t ) - {\hat{Z}}_{k} ( t ) \vert \qquad ( 7 )$$

However, it is believed that it is preferable to use a non-linear calibration model, preferably one established using a supervised artificial-intelligence algorithm.
Step 230: Using the calibration model.

After the calibration model (schematically shown in FIG. 4F) has been determined (cf. step 220), it is implemented to estimate reference measurements Z(t), at a measurement time t, on the basis of measurements X(t) acquired by the measurement sensor. The quantities Z(t) and X(t) have the same dimension. They are vector or scalar quantities. When the calibration model is used, the input layer of the model is X(t), whereas the output layer is Z(t).

### EXAMPLES

Experimental examples of processing of measurements, acquired by motion sensors, by a calibration model such as described with reference to FIG. 4F are described below.

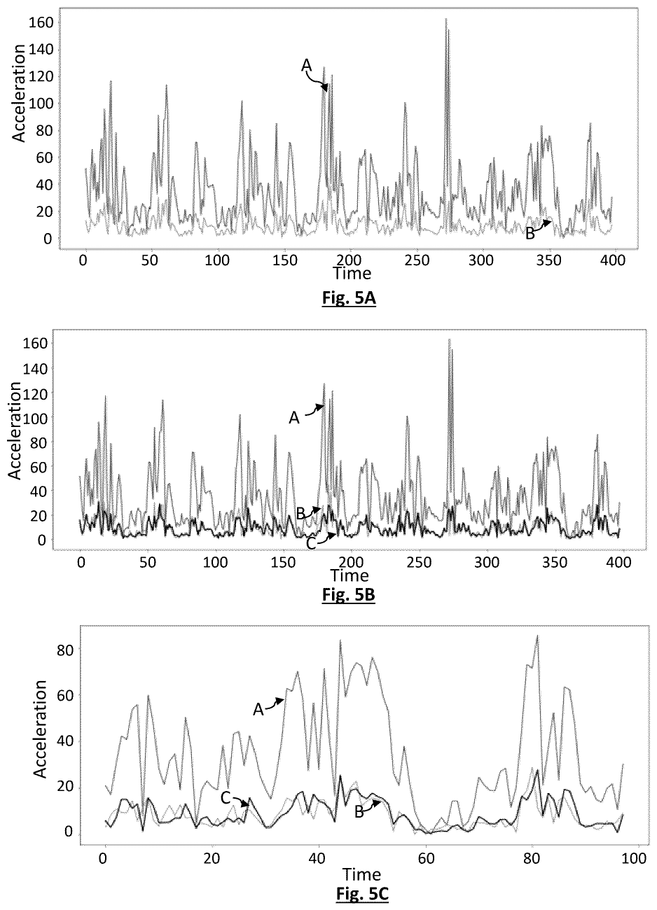

In a first series of trials, two identical accelerometer-comprising motion sensors were used (sensor A and sensor B). The sensors used were ST LSM6DSM inertial measurement units for mobile telephones (manufacturer ST Microelectronics). The firmware of each sensor was configured so that the low-pass filters were parametrized differently. The sensors were placed on the same wrist of a user. FIG. 5A shows one-axis acceleration measurements acquired at various measurement times. Sensor A was considered to be a reference sensor. Sensor B was considered to be the sensor needing to be calibrated. The measurements shown in FIG. 5A were used as calibration measurements (sensor B) and reference measurements (sensor A) to establish the calibration model.

In this example, and non-limitingly, the calibration model was such that:
the first neural network RNN1 comprised one hidden layer, comprising 50 neurons;
the second neural network RNN2 comprised one hidden layer, comprising 50 neurons;
the third neural network NN3 comprised one hidden layer, comprising 3 neurons.

FIG. 5B shows:
reference measurements, acquired by sensor A (curve A);
measurements acquired by sensor B to be calibrated (curve B);
measurements acquired by sensor B and processed by the calibration model (curve C).

The consistency between the reference measurements (curve A) and the estimates thereof by the calibration model (curve C) may be seen.

FIG. 5C is a detail of FIG. 5B, in the time range 300 s-400 s.

In a second series of trials, two different measurement sensors were used: a geneactiv sensor (GE)—manufacturer Active Insights (GE) and an Apple Watch—manufacturer Apple (AW). At various times, a tri-axis acceleration vector was measured. In the following, the reference sensor was the sensor GE. The sensor AW was the sensor being calibrated.

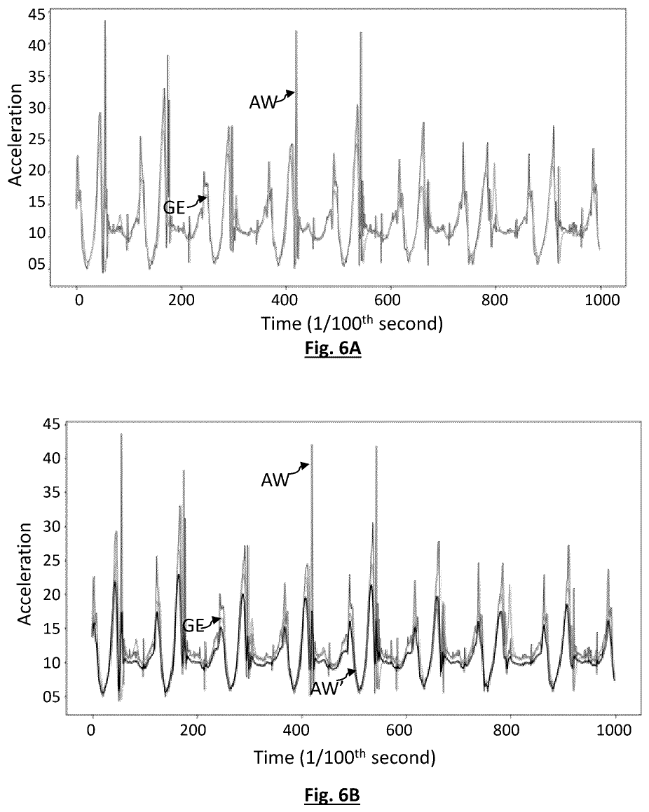

FIG. 6A shows one-axis acceleration measurements acquired at various measurement times. The sensors were placed on the wrist of a user, and the measurements taken while the latter was walking. The measurements shown in FIG. 6A were used to establish the calibration model.

The calibration model was such that:
the first neural network RNN1 comprised two hidden layers, each hidden layer comprising 50 neurons;
the second neural network RNN2 comprised two hidden layers, each hidden layer comprising 50 neurons;
the third neural network NN3 comprised three hidden layers, each hidden layer comprising 20 neurons, 20 neurons and 3 neurons, respectively.

FIG. 6B shows:
reference measurements, acquired by the sensor GE (curve GE);
measurements acquired by the sensor AW to be calibrated (curve AW);
measurements acquired by the sensor AW and processed by the calibration model (curve AW′).

The root-mean-square error (RMSE) was calculated:
between the reference measurements (GE) and the measurements acquired by the sensor AW: RMSE=0.316;
between the reference measurements (curve GE) and their estimates by the calibration model (curve AW′): RMSE=0.139.

Comparison of these RMSEs shows that processing the data measured by the sensor AW made it possible to get significantly closer to the reference measurements, this attesting to the relevance of the embodiments of the disclosure.

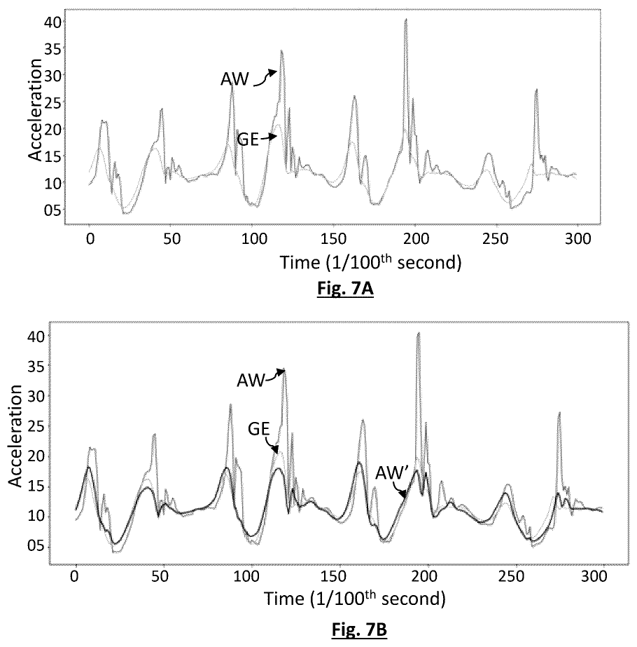

The sensors GE and AW were used to acquire measurements while the user was running. FIG. 7A shows one-axis acceleration measurements acquired at various measurement times. The measurements shown in FIG. 7A were used to establish the calibration model.

FIG. 7B shows:
reference measurements, acquired by the sensor GE (curve GE);
measurements acquired by the sensor AW to be calibrated (curve AW);
measurements acquired by the sensor AW and processed by the calibration model (curve AW′).

The root-mean-square error (RMSE) was calculated:
between the reference measurements and the measurements acquired by the sensor AW: RMSE=0.267;
between the reference measurements (curve GE) and their estimates by the calibration model (curve AW′): RMSE=0.126.

In another series of trials, an implementation of steps 100 to 150 described with reference to FIG. 3A was tested. Inertial measurement units (IMUs) of reference iNEMO (supplier ST Micro) were used as sensors. Each inertial measurement unit was mounted on a strap, fastened to the wrist of a test user. Each inertial measurement unit comprised an accelerometer, and registers allowing values of the parameters of the accelerometer to be adjusted. More precisely, the following were used:
a reference device, comprising an inertial measurement unit the registers of which were parametrized to correspond to a reference configuration;
a test device, comprising another inertial measurement unit, identical to the preceding one, the registers of which were parametrized differently to the reference configuration.

The test device and the reference device were worn on the same wrist of the same user.

Among the registers linked to the accelerometer, certain registers allowed a filter applied to the measurements delivered by the accelerometer to be configured. The parameters were:
HP_SLOPE_XL_EN: when the value is set to 0, the filter is a low-pass filter. When the value is set to 1, the filter is a high-pass filter.
LPF2_XL_EN, LPF1_BW_ESL and HPCF_XL: these registers allowed filtering parameters, and more precisely the cut-off frequency, to be defined. Cut-off frequency is determined based on the output data rate (ODR), i.e., sampling frequency.

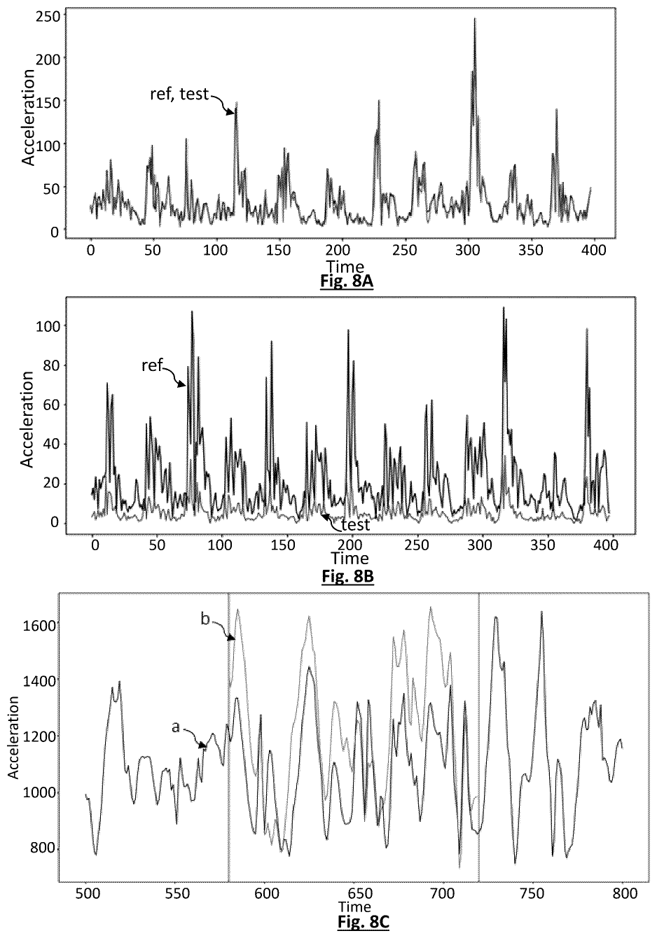

FIG. 8A shows measurements taken on the user, while the latter was walking. The reference device (ref) and the test device (test) were parametrized according to the reference configuration. The x-axis corresponds to time (units of seconds) and the y-axis corresponds to the acceleration measurements (units of mg where g corresponds to the magnitude of the acceleration due to gravity). Table 1 shows the value of each parameter in the reference configuration (cf. second column). The symbol “-” means the value was immaterial.

TABLE 1

| Register | Reference value | Modified value |
|---|---|---|
| HP_SLOPE_XL_EN | 1 | 1 |
| LPF2_XL_EN | 0 | 1 |
| LPF1_BW_ESL | — | — |
| HPCF_XL | — | 01 |
| Cut-off frequency | ODR/2 | ODR/100 |

In FIG. 8A, the curves “ref” and “test,” corresponding to the reference device and to the test device, respectively, overlap.

FIG. 8B represents measurements taken on the user, the latter continuing to walk while wearing the reference device and the test device. The configuration of the latter, i.e., the parametrization of the registers, was modified, resulting in a modification of the cut-off frequency. Cf. right-hand column of Table 1. In FIG. 8B, the x-axis corresponds to time (units of seconds) and the y-axis corresponds to the acceleration measurements (units of mg where g corresponds to the magnitude of the acceleration due to gravity).

The root-mean-square errors (RMSE) of the measurements shown in FIGS. 8A and 8B were calculated. The RMSEs were equal to 9 mg and 25.1 mg, respectively. Comparison of FIGS. 8A and 8B clearly shows the effect, on the measurements, of incorrect parametrization of the measurement sensor. This is what the embodiments of the disclosure make possible to solve.

FIG. 8C is a simulation of implementation of the embodiments of the disclosure. The y-axis corresponds to acceleration (units of mg). The x-axis corresponds to time (units of milliseconds). Curve a) is representative of nominal operation of the sensor, i.e., when it is parametrized with specified values. Between t=580 ms and t=720 ms, untimely maladjustment of the value of a parameter was simulated: whereas beforehand the sensor was correctly parametrized, the value of one sensor parameter was modified at 580 ms. The parameter value was verified at t=720 ms. Since the verification was non-conforming, the parameter value was corrected, so as to return the parameter to the initial value. The period comprised between 580 ms and 720 ms shows the effect of untimely maladjustment of a parameter value: cf. curve b). FIG. 8C shows the effect of the embodiments of the disclosure, before and after 720 ms. The embodiments of the disclosure allow the incorrect value of the parameter to be reset to the initial value, this leading to measurements representative of the nominal operation of the sensor.

The embodiments of the disclosure allow measurements representative of a variation as a function of time in a physical quantity to be obtained, independently of the sensors allowing the measurements to be acquired. The measurements, after the calibration such as described above, are comparable with one another, or “standardized.” They may be used by a given interpreting application.

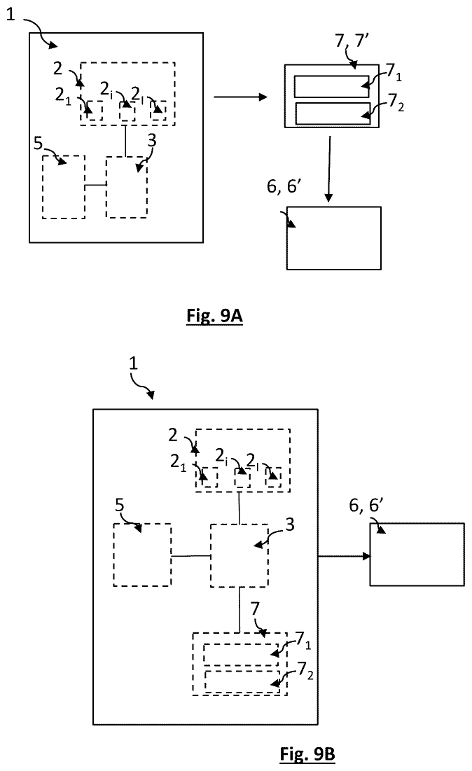

In the above examples, the interfacing application 7 is implemented by a microprocessor, in fact a central unit 5 integrated into the same device 1 as the measurement sensor. According to another embodiment, the interfacing application 7 is implemented by a remote microprocessor. According to this embodiment, which has been shown in FIG. 9A, the data measured by the measurement sensor 2 are transmitted to a remote microprocessor 7′, implementing the interfacing application. The interfacing application 7 may be implemented in a cloud server, i.e., a server accessible over the Internet. The reference measurements delivered by the interfacing application are then transmitted to another remote microprocessor 6′, implementing the interpreting application 6. Similarly to the interfacing application 7, the interpreting application may be implemented in a cloud server, i.e., a server accessible over the Internet.

In this case, the measurements transmitted by the measurement sensor include an identifier, making it possible for the interfacing application 7 to identify the measurement sensor and to apply a calibration model that corresponds to the latter, and that was established beforehand in a calibrating phase and stored in a memory connected to the remote microprocessor 7′.

According to another embodiment, the interfacing application is implemented in the same device as that comprising the measurement sensor. The interpreting application is implemented by a remote microprocessor 6′. Such a variant has been shown in FIG. 9B.

What is claimed is:

1. A device, comprising:
a measurement sensor, configured to acquire raw measurements, each raw measurement representing a movement of the user or a physiological characteristic of the user; and
an electronic acquisition circuit, connected to the measurement sensor, and configured to control acquisition of the raw measurements by the measurement sensor, and/or configured to pre-process the raw measurements, so that each raw measurement or each pre-processing of the raw measurements is controlled by an acquisition parameter, wherein the acquisition parameter is stored in one hardware control register physically implemented in the electronic acquisition circuit, wherein a specified value is assigned to the acquisition parameter;
wherein:
the device is worn by a user;
the device is connected to a central processing unit;
the central processing unit is configured to receive raw measurements or pre-processed raw measurements, so as to estimate a state of the user;
the central processing unit is configured to periodically:
read a stored value of the acquisition parameter in the hardware control register;
compare the stored value of the acquisition parameter with the specified value assigned to the acquisition parameter;
determine a conformity of the acquisition parameter with respect to the specified value assigned to the acquisition parameter, so that the verification is:
negative when a value of the acquisition parameter does not conform with the value assigned to the acquisition parameter;
positive when a value of the acquisition parameter does conform with the value assigned to the acquisition parameter;
following a negative verification, replace the stored value of the acquisition parameter, in the hardware control register, with the specified value assigned to the acquisition parameter.

2. The device of claim 1, wherein the central processing unit is configured to:
successively verify the conformity of the acquisition parameter, during the acquisition of raw measurements;
following a negative verification, labelling the raw measurements acquired since a preceding positive verification and/or until a following positive verification as doubtful or invalid.

3. The device of claim 1, wherein the central processing unit is configured to compare the successive raw measurements or the pre-processed raw measurements to a standard sequence of measurements, wherein said standard sequence is stored in a memory connected to the central processing unit.

4. The device of claim 1, wherein the central processing unit is configured to:
process the raw measurements or the pre-processed raw measurements by means of a calibration model, so as to estimate, on the basis of each of the raw measurements or the pre-processed raw measurements, a reference measurement;
estimate the state of the user using said reference measurement;
wherein the calibration model is established during a calibrating phase, comprising various calibration times, the calibrating phase comprising:
i) acquiring calibration measurements using the measurement sensor, at the various calibration times, and obtaining reference measurements, at each calibration time, such that each reference measurement corresponds to a calibration measurement, at least one reference measurement being representative of a user state among predetermined states;
ii) on the basis of the reference measurements and of the calibration measurements, defining a calibration model, the calibration model being configured to estimate reference measurements on the basis of measurements acquired by the measurement sensor;
wherein each reference measurement is an acquisition of the same physical or chemical quantity as each calibration measurement.

5. The device of claim 4, wherein, in step i), the measurement sensor is placed on a phantom, representative of at least one user state, the reference measurements being obtained from the phantom.

6. The device of claim 5, wherein the phantom comprises a reference sensor, of the same type as the measurement sensor, the reference sensor delivering a reference measurement at each calibration time.

7. The device of claim 4, wherein, in i), a calibration sensor is placed on at least one test individual, the test individual also wearing/bearing a reference sensor, the reference sensor delivering a reference measurement at each calibration time, wherein the reference sensor is of the same type as the measurement sensor.

8. The device of claim 7, wherein the calibration model implements a supervised artificial-intelligence algorithm, the supervised artificial-intelligence algorithm being parametrized in the course of the calibrating phase.

9. The device of claim 8, wherein the supervised artificial-intelligence algorithm comprises a recurrent neural network, the recurrent neural network comprising an input layer and an output layer, such that during the processing of each of the raw measurements or pre-processed raw measurements by the calibration model:
each of the raw measurements or pre-processed raw measurements acquired by the measurement sensor is provided to the input layer of the neural network;
the output layer comprises an estimated reference measurement.

10. The device of claim 4 comprising, between steps i) and ii), time synchronizing of the calibration measurements with respect to the reference measurements.

11. The device of claim 1, wherein the measurement sensor comprises at least:
a motion sensor, of the accelerometer and/or gyrometer and/or magnetometer type; and/or
a pressure sensor; and/or
an optical sensor; and/or
an electrical sensor; and/or
a chemical sensor; and/or
a temperature sensor; and/or
a physiological sensor, configured to determine a physiological characteristic of the user.

12. The device of claim 1, wherein the state of the user is selected from:
a state describing a physical activity of the user;
a state of stress;
a state of sleep or drowsiness;
a pathological state;
a symptomatic state;
a state corresponding to occurrence of a situation putting the user at risk.

13. The device of claim 1, wherein the central processing unit comprises a microprocessor integrated into the device, or a microprocessor remote from the device and connected to the latter by a wired or wireless link.
